(19) United States
(12) Patent Application Publication
(10) Pub. No.: US 2007/0109116 A1
(43) Pub. Date: May 17, 2007

(54) Wireless power source and/or communication for bioarrays

(76) Inventor: Jeremy Burr, Portland, OR (US)

Correspondence Address: INTEL CORPORATION;c/o INTELLEVATE, LLC, P.O. BOX 52050, MINNEAPOLIS, MN, 55402, US

(21) Appl. No.: 11/273,810

(22) Filed: Nov. 14, 2005

(51) Int. Cl. G08B 1/08; A61B 5/00

(52) U.S. Cl. 340/539.12; 600/300

(57) ABSTRACT

In various embodiments, a bio-op device (a device for performing biological operations of detection and/or transport and/or synthesis of biological molecules) may have either or both of the following characteristics: 1) it uses electrical energy harvested from electromagnetic (EM) radiation received by the bio-op device to power the operations of the bio-op device, 2) it reports operation results wirelessly using a radio circuit that is part of, or attached to, the bio-op device.

### BACKGROUND

It is possible to dynamically perform biomolecular transport, detection and synthesis on a substrate surface under electronic control. These devices and processes may be dynamically reconfigured to perform multistep and combinatorial reactions at micro-locations upon the substrate surface. The microfluidic collection, separation, and channel transfer of biomolecules such as DNA can be electronically controlled using digital logic microelectrodes. The electronically charged biomolecules can be manipulated by digitally controlled electric fields with standard logic levels. These microarray devices are able to create reconfigurable electric field transport geometries on their surface which allows charged reagent and analyte molecules (e.g., DNA, RNA, oligonucleotide probes, amplicons antibodies, proteins, enzymes, nanoparticles and micro sized semiconductor devices) to be moved to or from the various microscopic test sites on the device surface. Biomolecule synthesis may also be conducted on microelectronic devices with programmable and addressable microscopic locations. For all their capabilities, however, these devices are generally confined to restricted usage environments because they must be controlled and report their results through a wired connection, and they must be connected to a power source through a wired power connection or through an on-board battery. This greatly limits the environments in which they may operate.

### BRIEF DESCRIPTION OF THE DRAWINGS

Some embodiments of the invention may be understood by referring to the following description and accompanying drawings that are used to illustrate embodiments of the invention. In the drawings:

FIG. 1 shows a bio-op system, according to an embodiment of the invention.

FIG. 2 shows a bio-op device, according to an embodiment of the invention.

FIG. 3 shows a flow diagram of a method of operation of a bio-op device, according to an embodiment of the invention.

FIG. 4 shows a flow diagram of a method of operation of a device communicating with a bio-op device, according to an embodiment of the invention.

FIG. 5 shows multiple bio-op devices on a single substrate, according to an embodiment of the invention.

### DETAILED DESCRIPTION

In the following description, numerous specific details are set forth. However, it is understood that embodiments of the invention may be practiced without these specific details. In other instances, well-known circuits, structures and techniques have not been shown in detail in order not to obscure an understanding of this description.

References to “one embodiment”, “an embodiment”, “example embodiment”, “various embodiments”, etc., indicate that the embodiment(s) of the invention so described may include particular features, structures, or characteristics, but not every embodiment necessarily includes the particular features, structures, or characteristics. Further, some embodiments may have some, all, or none of the features described for other embodiments.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements co-operate or interact with each other, but they may or may not be in direct physical or electrical contact.

The term “processor” may refer to any device or portion of a device that processes electronic data from registers and/or memory to transform that electronic data into other electronic data that may be stored in registers and/or memory. A “computing platform” may comprise one or more processors.

Within the context of this document, an RFID tag may be defined as comprising an RFID antenna (to receive an incoming signal that serves to query the RFID tag and to transmit a response in the form of a modulated radio frequency signal), and an RFID tag circuit (which may include circuitry to store an identification code for the RFID tag, circuitry to transmit that code through the antenna, and in some embodiments a power circuit to collect received energy from the incoming radio frequency signal and provide that energy to power the operations of the RFID tag circuit). As is known in the field of RFID technology, “transmitting” a signal from an RFID tag may include either: 1) providing sufficient power to the antenna to generate a signal that radiates out from the antenna, or 2) reflecting a modulated version of the received signal. The term “wireless” and its derivatives may describe communications using electromagnetic waves traveling through a non-solid medium, but does not imply whether the associated devices do or don't have any wires.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Various embodiments of the invention may be implemented in one or any combination of hardware, firmware, and software. The invention may also be implemented as instructions contained in or on a machine-readable medium, which may be read and executed by one or more processors to perform the operations described herein. A machine-readable medium may include any mechanism for storing, transmitting, and/or receiving information in a form readable by a machine (e.g., a computer). For example, a machine-readable medium may include a storage medium, such as but not limited to read only memory (ROM); random access memory (RAM); magnetic disk storage media; optical storage media; flash memory devices. A machine-readable medium may also include a tangible medium through which electrical, optical, acoustical or other form of propagated signals representing the instructions may pass, such as antennas, optical fibers, communications interfaces, and others.

Various embodiments of the invention may pertain to a biological operations (bio-op) device that has either or both of the following characteristics: 1) it uses the electrical energy harvested from received electromagnetic (EM) radiation to power the operations of the bio-op device, 2) it reports operation results wirelessly with a radio circuit that is part of, or is attached to, the bio-op device. A bio-op device, as the term is used herein, is a device that can perform operations of detection and/or transport and/or synthesis of biological molecules. When used together, the two listed characteristics may permit the bio-op device to operate untethered by wires to any external device such as a computer or a power source, thus freeing it up for in situ use (such as in a long-term sealed environment), and may even permit use deep inside a living organism. Some embodiments may use radio frequency identification (RFID) technology, using an RFID tag circuit as a wireless communications receiver or transceiver, and may also use RFID technology for wireless power harvesting. In some embodiments, the operations may include dynamic reconfiguration of a bioarray, so that various operations of detection and/or transport and/or synthesis may all be performed automatically by a single bio-op device while remaining on site, without direct human intervention.

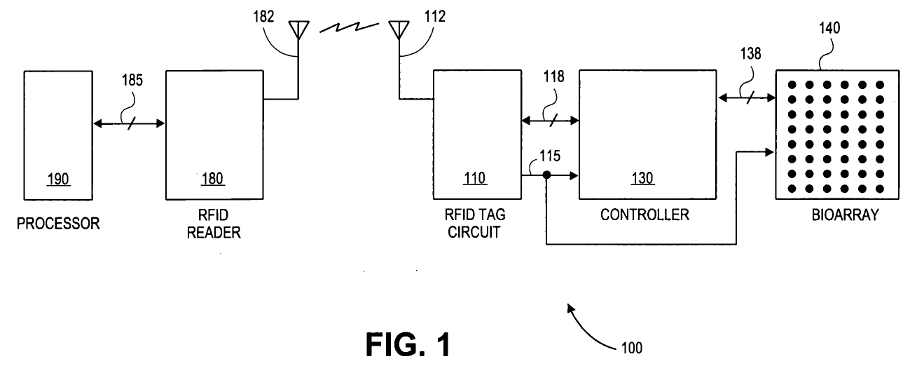

FIG. 1 shows a bio-op system, according to an embodiment of the invention. In the illustrated example, the system comprises bio-op device 100, a radio frequency identification (RFID) reader 180, and in some embodiments a processor 190. Bio-op device 100 may comprise a bioarray 140, a controller 130 to control operations of the bioarray, and a radio frequency identification (RFID) tag that contains an RFID circuit 110 and an antenna 112. Bioarray 140 may contain an arrangement of multiple tiny closely-spaced electrodes, which can individually be accurately charged to very small voltage levels, and that are arranged on the surface of a semiconductor device. In some embodiments, from 100 to 1000 of the electrodes may be arranged in a matrix configuration, but other embodiments may use different quantities and/or different arrangements of the electrodes. The device may be configured so that the surface containing the electrodes may come into direct physical contact with the biological solution or material that is being operated upon. The particular operations that may be performed by bioarray 140 may be known in the art and are not described herein to avoid obscuring an understanding of the various embodiments of the invention.

Controller 130 may be used to control the bioarray 140, and detect the results of the operations of the bioarray 140, through signal connections 138. Controlling bioarray 140 may include configuring and/or reconfiguring the bioarray (such as, but not necessarily limited to, changing the amount of charge on the various electrodes) for different operations. An RFID tag (containing the RFID circuit 110 and antenna 112) may perform two-way wireless communications between the bio-op device 100 and an RFID reader 180. In some embodiments the RFID tag circuit 110, the controller 130, and the bioarray 140 may all be contained in the same integrated circuit die. In some other embodiments any two or three of these three items (i.e., RFID tag circuit 110, controller 130, and bioarray 140) may be contained in the same integrated circuit package but distributed among two or more integrated circuit dice. Other embodiments may use other arrangements for physical packaging. The RFID reader 180 may transmit a signal through antenna 182 to the RFID tag which is received through antenna 112. As is known in the field of RFID technology, the RFID tag may ‘harvest’ (i.e., accumulate) electrical power from the received signal and use that energy to power the RFID circuit 110. The RFID tag may then transmit its own identification number plus additional information back to the RFID reader 180. This additional information may include data that represents the results of various operations performed by the bioarray 140. The RFID tag may communicate with controller 130 through signal connections 118.

In some embodiments the RFID reader 180 may transmit information to the RFID tag by modulating the aforementioned transmitted signal from the RFID reader. Such information may include one or more of such things as: 1) an address or other identifier so that the RFID tag knows it is the intended recipient of the transmission from the RFID reader 180, 2) other data that indicates how the RFID tag is to respond, and 3) commands to the bio-op device 100. Such commands may include, but are not limited to, such things as a) configure the bioarray in a specified way, b) report results of the operations, c) control power to the controller and/or bioarray, d) etc.

In the illustrated embodiment, the electrical power harvested by the RFID tag may also be used to power the controller 130 and/or bioarray 140, through connection 115. In some embodiments, the RFID tag may be able to switch this power to the controller 130 and/or bioarray 140 off or on, so that the RFID tag may communicate with the RFID reader 180 without powering up all the available functions.

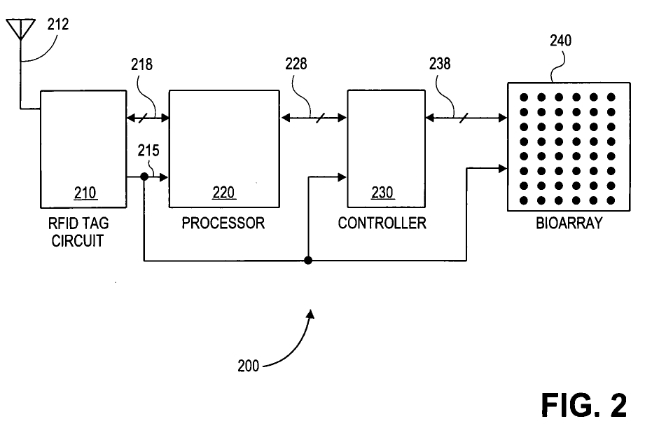

To be useful, the results of these operations may need to be processed and analyzed. Such processing may also determine which operation is to be performed next, and therefore determine how to reconfigure the bio-array 140. In some embodiments the bio-op device 100 may not have enough processing capacity to perform such processing, and may simply pass the operation results back to the RFID reader 180, which may contain sufficient processing power or may pass the results on to another processor 190 through data path 185 for processing. Processor 190 may be located locally or at a distance from RFID reader 180, and data path 185 may involve using any feasible type of communications with the RFID reader 180. However, in some embodiments, if the bio-op device has sufficient processing power, all or a portion of such processing may be performed locally within the bio-op device, such as shown in FIG. 2.

FIG. 2 shows a bio-op device, according to an embodiment of the invention. In the illustrated example, bio-op device 200 comprises a bioarray 240, a controller 230 to control operations of the bioarray, and a radio frequency identification (RFID) tag that contains an RFID circuit 210 and an antenna 212, as well as connections 215, 218, and 238. In some embodiments these may correspond to their similarly numbered counterparts 140, 130, 110, 112, 115, 118 and 138 in FIG. 1. However, the embodiment of FIG. 2 may also include additional local processing power in the form of processor 220, which may communicate with controller 230 through connections 228 and RFID tag circuit 210 through connections 218. Although shown as being coupled between the RFID tag circuit 210 and controller 230, the processor 220 may be coupled in any feasible manner. Processor 220 may also include memory and other circuitry as needed to perform its purpose.

Although the embodiments of FIGS. 1 and 2 show an RFID tag providing the electrical power for the bio-op device and also providing two-way communications, other embodiments may use other techniques. For example, some embodiments may use an on-board battery to provide power to the bioarray and/or controller. Some embodiments may use electromagnetic induction to provide wireless power to the bio-op device. Some embodiments may use solar cells or other photo-voltaic components to provide power to the bioarray and/or controller. Some embodiments may use other energy collection techniques that convert specific portions of the electromagnetic spectrum into usable electrical energy. Some embodiments may use a conventional battery-powered radio receiver (to report results) or transceiver (to receive commands and report results) circuit in place of the RFID tag circuit to communicate with external devices. In such an embodiment, a conventional radio may be used in place of RFID reader 180. These and other techniques may be used in various combinations. Although not specifically described in the drawings, these forms of communication and energy transmittal/conversion are sufficiently understood by those of ordinary skill in the art to be implemented without describing the details of their implementation in the drawings.

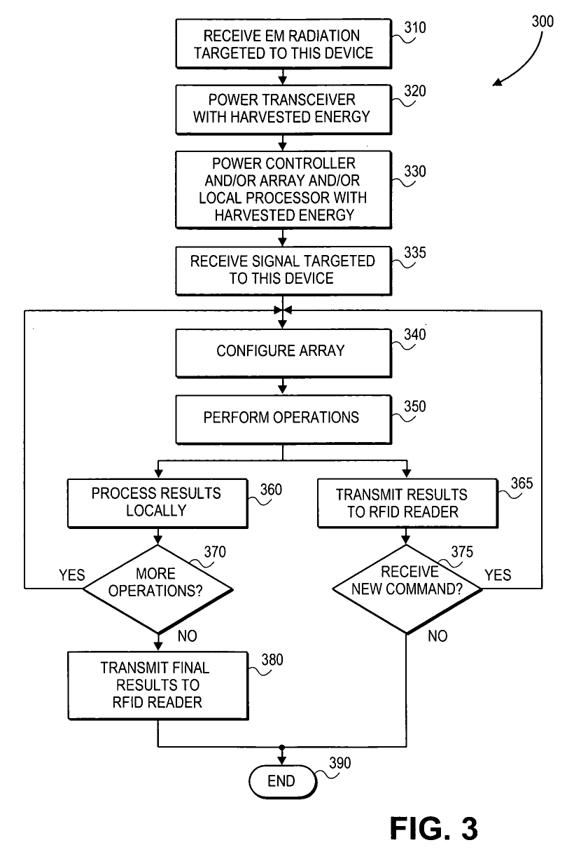

FIG. 3 shows a flow diagram of a method of operation of a bio-op device, according to an embodiment of the invention. In flow diagram 300, at 310 the bio-op device may receive electromagnetic radiation targeted to it. In some embodiments such targeting may be in the form of a radio frequency signal within the right frequency band. In other embodiments such targeting may be in the form of 60 Hz electromagnetic induction in close proximity. Still other embodiments may use visible light directed onto photo-voltaic cells on the bio-op device. Other techniques may also be used. At 320 energy from the received EM radiation may be harvested and used to power operation of a radio transceiver. In embodiments using harvested energy for the controller and/or bio-array and/or local processor, at 330 those devices may be powered with that harvested energy. At 335, a wireless signal may be received by the bio-op device, the wireless signal modulated with a destination address that matches an internal address of the bio-op device or a component of it. The signal may also contain other information for the bio-op device (e.g., a command, configuration data, etc.). In some embodiments using RFID technology, an RFID tag may be used to harvest the received energy and receive the address/command/data/etc.

In embodiments with a reconfigurable array, the bio-array may be configured at 340 based on command(s) received at 335. One or more transmissions may be required to communicate the command(s), depending on the specifics of the communications system being used. Once the bio-array has been configured, the operations that are enabled by that configuration may be performed at 350, with the subject biological material in contact with the bio-array. The time that this takes may depend on the type of operation being performed.

What follows next may depend on whether the bio-op device has on-board processing power. If it does, the results of the preceding operation may be processed at 360. A determination is then made at 370 whether additional operations are to be performed. If so, the bio-array may be re-configured at 340 and the next operation performed. This sequence may be repeated until there are no more sequential operations to be performed, in which case the results may be transmitted back to an external device (such as an RFID reader) at 380. Intermediate results may also be transmitted at various times, such as after 360. The process may be terminated at 390, in some embodiments by removing power from the bio-array and/or controller.

If the bio-op device does not have sufficient on-board processing power, then the results of the operations at 350 may be transmitted to an external device (such as an RFID reader) at 365. The external device, or a device in communication with it, may do the processing and decide whether another operation needed. If so, the external device may transmit the proper command(s) to the bio-op device at 375, triggering a reconfiguration at 340 and another operation. This sequence may continue until there are no more operations to be performed. At that point, the process may be terminated at 390 as previously described.

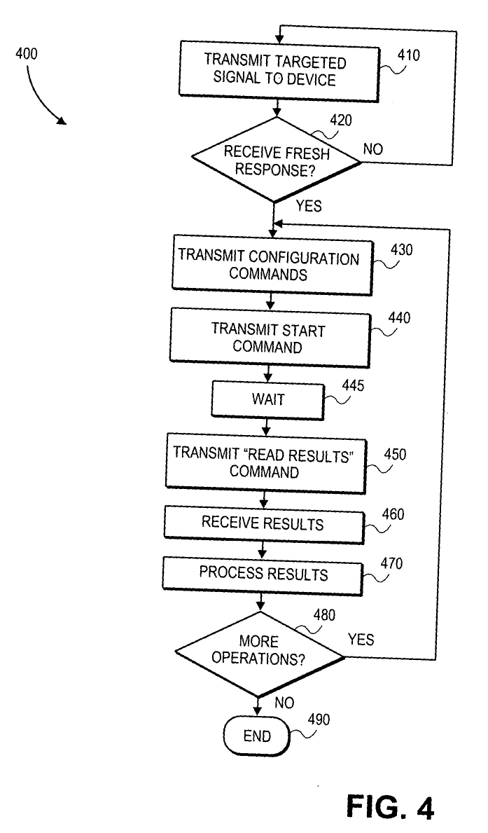

FIG. 4 shows a flow diagram of a method of operation of a device communicating with a bio-op device, according to an embodiment of the invention. In flow diagram 400, at 410 a signal may be transmitted that is targeted at a specific bio-op device. The various techniques of targeting may be as previously described for FIG. 3. At 420 a signal may be received from the targeted device that acknowledges the signal transmitted at 410 was received. In some types of RFID technology, the RFID tag may respond multiple times. If so, the decision block at 420 may be used to eliminate redundant responses by looping at 410-420 until the first (i.e., a ‘fresh’) response is received, and then continuing on at 430. In embodiments that involve reconfigurable bio-arrays, configuration command(s) may be transmitted at 430. In embodiments that require a start signal after the configuration is complete, a start command may be transmitted to the bio-device at 440.

A period of waiting is shown at 445. The duration of this wait may be determined by various factors, such as but not limited to: 1) a timer set to expire after a pre-determined period that is judged to produce viable results in the current operation of the bio-op device, 2) a communication from the bio-op device that it has results ready, 3) intervention by another device, 4) human intervention, 5) etc. After the wait period, at 450 a command may be transmitted to the bio-op device instructing it to transmit the results of the current operation, and at 460 those results may be received. At 470 those results may be processed, either in the receiving device or by another device that the results have been forwarded to. A determination may then be made at 480 whether another operation is needed. If so, new configuration command(s) may be transmitted to the bio-op device at 430 and the sequence repeats itself until no more operations are needed. Once all operations have been completed as determined at 480, the process may be terminated at 490. Termination may include removing operational power from the bio-op device.

The embodiments of the aforementioned flow diagrams describe particular operational elements. Other embodiments may use fewer, more, and/or different operational elements than described.

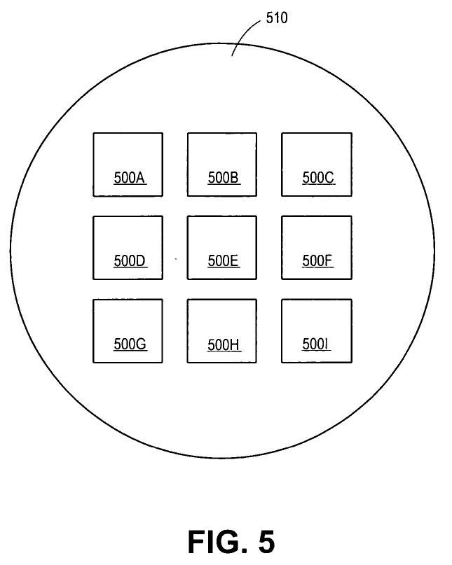

FIG. 5 shows multiple bio-op devices on a single substrate, according to an embodiment of the invention. Nine bio-op devices are shown, but any feasible number may be included on the same substrate. In the illustrated example, each of the nine bio-op devices 500A-I may be similar to bio-op device 100 or 200 previously described. They may all be fabricated on a single substrate 510. In some embodiments, substrate 510 may be a wafer of silicon, gallium arsenide, or other material as is commonly used as a substrate in the manufacturing of integrated circuits. In some embodiments, all the bio-op devices 500A-5001 may be manufactured substantially identical to each other except for the communications address contained within each one, while in other embodiments there may be multiple versions of the bio-op devices on the same substrate. Having multiple bio-op devices located together on a single substrate can substantially increase the number of bioarrays that may be used at the same time, while preserving the ability to perform different operations on each at the same time. In addition, the bio-op devices may later be separated into multiple separate bio-op devices by cutting the substrate into multiple pieces. In a somewhat different embodiment, each device 500x may contain only the controller and bioarray, and a single radio circuit may be used to control the operations of multiple bioarrays by connected it to each device 500x.

The foregoing description is intended to be illustrative and not limiting. Variations will occur to those of skill in the art. Those variations are intended to be included in the various embodiments of the invention, which are limited only by the

What is claimed is:

1. An apparatus, comprising:
a first bioarray;
a controller coupled to the first bioarray to control an operation of the first bioarray; and
a radio circuit coupled to the controller to wirelessly transmit a result of the operation of the first bioarray.

2. The apparatus of claim 1, wherein the radio circuit is to wirelessly receive at least one command pertaining to the operation of the first bioarray.

3. The apparatus of claim 2, wherein the operation is a configuration operation.

4. The apparatus of claim 1, wherein the radio circuit comprises a first radio frequency identification (RFID) tag circuit, the first RFID tag circuit to:
power itself from radio frequency (RF) energy received through an RFID antenna; and
wirelessly transmit the result through the RFID antenna.

5. The apparatus of claim 4, wherein the first RFID tag circuit is to power the operation of the first bioarray from the RF energy received through the RFID antenna.

6. The apparatus of claim 4, wherein the first bioarray and the first RFID tag circuit are in a same integrated circuit die.

7. The apparatus of claim 4, wherein the first bioarray and the first RFID tag circuit are in a same integrated circuit package.

8. The apparatus of claim 4, further comprising a second bioarray electrically coupled to a second RFID tag circuit, wherein the first and second bioarrays and the first and second RFID tag circuits are on a same substrate.

9. The apparatus of claim 1, further comprising an antenna coupled to the radio circuit.

10. A method, comprising:
receiving electromagnetic radiation;
harvesting electrical energy from the received electromagnetic radiation;
powering a bioarray with the harvested electrical energy;
performing an operation with a bioarray; and
wirelessly transmitting a result of the operation.

11. The method of claim 10, further comprising:
wirelessly receiving a command for the bioarray through an antenna; and
configuring the bioarray based on the command.

12. The method of claim 10, wherein said receiving, said harvesting, and said powering are performed with a radio frequency identification (RFID) tag.

13. The method of claim 10, wherein said performing an operation and said transmitting are performed within a living organism.

14. The method of claim 10, wherein said performing the operation comprises performing an operation selected from a list consisting of: molecular detection, molecular transport, and molecular synthesis.

15. An apparatus, comprising
a radio frequency identification (RFID) reader device to:
wirelessly transmit a first signal targeted to a bio-op device; and
wirelessly receive a second signal from the bio-op device modulated with a result of a first operation performed by the bio-op device.

16. The apparatus of claim 15, wherein the RFID reader is to forward the result to a processor for analysis.

17. The apparatus of claim 15, wherein the RFID reader device is to process the result for analysis.

18. The apparatus of claim 15, wherein the RFID reader device is to wirelessly transmit a third signal targeted to the bio-op device to command the bio-op device to be reconfigured for a second operation.

19. The apparatus of claim 15, further comprising an antenna coupled to the RFID reader device.

20. A method, comprising:
wirelessly transmitting a first signal targeted to a bio-op device; and
wirelessly receiving a second signal from the bio-op device modulated with a result of a first operation performed by the bio-op device.

21. The method of claim 20, further comprising forwarding the result to a processor for analysis.

22. The method of claim 20, further comprising wirelessly transmitting a third signal targeted to the bio-op device to command the bio-op device to be reconfigured for a second operation.

23. The method of claim 20, further comprising processing the result for analysis.

24. The method of claim 20, wherein said wirelessly transmitting comprises wirelessly transmitting the first signal to a radio frequency identification (RFID) tag.

25. The method of claim 20, wherein the first operation is an operation selected from a list consisting of: molecular detection, molecular transport, and molecular synthesis.

26. An article comprising
a machine-readable medium that contains instructions, which when executed by at least one machine results in performing:
transmitting a first wireless signal targeted to a bio-op device; and
receiving a second wireless signal from the bio-op device modulated with a result of a first operation performed by the bio-op device.

27. The article of claim 26, wherein said executing the instructions further results in performing forwarding the result to a processor for analysis.

28. The article of claim 26, wherein said executing the instructions further results in performing transmitting a third wireless signal targeted to the bio-op device to command the bio-op device to be reconfigured for a second operation.

29. The article of claim 26, wherein said executing the instructions further results in performing processing the result for analysis.

30. The article of claim 26, wherein said transmitting a first wireless signal comprises transmitting the first wireless signal to a radio frequency identification (RFID) tag in the bio-op device.
